(19) United States
(12) Patent Application Publication
(10) Pub. No.: US 2021/0218998 A1
(43) Pub. Date: Jul. 15, 2021

(54) Methods for Reconstruction Coupled, Fast and Memory Efficient Visualization for High Dimensional Medical Image Datasets

(71) Applicants: The Board of Trustees of the Leland Stanford Junior University, Stanford, CA (US); The Regents of the University of California, Oakland, CA (US)

(72) Inventors: Frank Ong, Los Altos, CA (US); John M. Pauly, Stanford, CA (US); Shreyas S. Vasanawala, Stanford, CA (US); Michael Lustig, Moraga, CA (US); Nien Sin Cedric Yue Sik Kin, San Carlos, CA (US)

(21) Appl. No.: 17/146,355

(22) Filed: Jan. 11, 2021

Related U.S. Application Data
(60) Provisional application No. 62/959,923, filed on Jan. 11, 2020.

(51) Int. Cl. H04N 19/63

(52) U.S. Cl. CPC H04N 19/63

(57) ABSTRACT

A method of medical imaging includes performing a medical imaging scan to produce acquired imaging data; reconstructing from the acquired imaging data a multi-dimensional medical imaging dataset in the form of a sliceable compressed representation where the reconstruction does not at any stage create full decompressed images; and producing from the sliceable compressed representation a selected image slice by decompressing only a subset of the sliceable compressed representation. The sliceable compressed representation may be stored in a lossless format, and the selected image slice may be displayed on a viewer for visualization.

### CROSS-REFERENCE TO RELATED APPLICATIONS

This application claims priority from U.S. Provisional Patent Application 62/959,923 filed Jan. 11, 2020, which is incorporated herein by reference.

### STATEMENT REGARDING FEDERALLY SPONSORED RESEARCH OR DEVELOPMENT

This invention was made with Government support under contract EB009690 awarded by the National Institutes of Health. The Government has certain rights in the invention.

### FIELD OF THE INVENTION

The present invention relates generally to techniques for medical imaging. More specifically, it relates to techniques for image reconstruction and visualization from multi-dimensional medical imaging datasets.

### BACKGROUND OF THE INVENTION

High-dimensional medical imaging has become an important component in many applications, such as dynamic contrast enhanced CT, volumetric flow MRI, and whole-body dynamic PET. Among its many benefits, multi-dimensional imaging offers high quality multiplanar and/or temporal reformatting, which can greatly enhance clinical interpretation. Recent advances in model-based reconstruction methods, such as compressed sensing [1,2], have further pushed the achievable resolution limits. In particular, these techniques are able to reconstruct large-scale datasets by enforcing low dimensional image representations.

On the other hand, visualizing and storing these large sets of images pose immense challenges to hospital computing backends. Datasets stored in Digital Imaging and Communications in Medicine (DICOM) formats often have sizes on the order of 10-100 GBs, which result in expensive storage costs and slow data transfer. Moreover, visualization consoles require massive amounts of memory just to load the images. This can cause latency in visualization longer than 10 seconds per reformat, which impedes radiologist workflow. While off-the-shelf lossless compression methods can reduce data sizes, they further increase loading times.

Several publications [3-5] have proposed fast and memory efficient visualization of large-scale datasets by accessing and decompressing relevant subsets of the compressed data. However, one drawback with these methods is that the full image dataset is still needed in the first place so that it can be converted into suitable compressed formats.

### BRIEF SUMMARY OF THE INVENTION

The present invention addresses a problem with existing approaches for efficient visualization and storage of large-scale datasets by accessing only relevant subsets of the compressed data. These existing methods require that the full image dataset is still needed in the first place so that it can be converted into suitable compressed formats. To overcome this drawback, the present invention provides a framework that couples reconstruction with efficient visualization of high-dimensional medical image datasets, so that the full images never need to be created or stored.

In particular, the framework directly reconstructs datasets in sliceable compressed representations (SCR), which allows for efficient decompression of an image slice from only subsets of the compressed data. Examples of SCR include wavelet transforms, and various low rank matrix and tensor based compressed representations. We then directly store SCR datasets in a lossless format. Reformatting and slicing these datasets can then be done without operating on the full image dataset.

As an example illustration, we demonstrate the effectiveness of the proposed framework on MRI datasets reconstructed with two low rank based reconstruction methods, T2 shuffling [6] and multi-scale low rank [7], and show that memory usage and latency can be reduced by more than 15 times. We note that our method can be generally applied to imaging modalities beyond MRI.

In one aspect, the invention provides improved techniques for the storage and visualization of large multi-dimensional medical imaging datasets. Image reconstruction is coupled with efficient visualization of high-dimensional medical image datasets, so that the full image sets never need to be created or stored. In particular, datasets are directly reconstructed in sliceable compressed representations (SCR), which allows for efficient decompression of an image slice from only subsets of the compressed data. The SCR datasets are then directly stored in a lossless format. Reformatting and slicing these datasets is performed without operating on the full image dataset.

In one aspect, the invention provides a method of medical imaging comprising: performing a medical imaging scan to produce acquired imaging data; reconstructing from the acquired imaging data a multi-dimensional medical imaging dataset in the form of a sliceable compressed representation where the reconstruction does not at any stage create full decompressed images; and producing from the sliceable compressed representation a selected image slice by decompressing only a subset of the sliceable compressed representation.

Producing from the sliceable compressed representation a selected image slice comprises may include selecting the subset of the sliceable compressed representation, and decompressing the subset to form the selected image slice.

The method may also include storing the sliceable compressed representation in a lossless format. The method may also include displaying the selected image slice on a viewer for visualization.

The sliceable compressed representation may be a wavelet transform compressed representation or a low rank matrix and tensor based compressed representation. The low rank matrix and tensor based compressed representation may be reconstructed using a T2 shuffling method or a multi-scale low rank method.

### BRIEF DESCRIPTION OF THE SEVERAL VIEWS OF THE DRAWINGS

FIG. 1 illustrates a processing pipeline for generating sliceable compressed representations from acquired multidimensional image data and visualizing selected slices, according to an embodiment of the invention.

FIG. 2 illustrates an image reconstruction pipeline that enforces sliceable compressed representations, according to an embodiment of the invention.

FIG. 3 illustrates a processing pipeline for generating from sliceable compressed representations decompressed sliced data for visualization, according to an embodiment of the invention.

FIG. 4 is an image illustrating an application of the SCR method on a knee MRI dataset, according to an embodiment of the invention.

FIG. 5 is an image illustrating an application of the SCR method on a chest DCE MRI dataset, according to an embodiment of the invention.

### DETAILED DESCRIPTION OF THE INVENTION

Sliceable Compressed Representation (SCR)

We broadly define and use sliceable compressed representations (SCR) for large-scale medical image datasets, which allow us to compress datasets and retrieve an image slice at a particular location efficiently. In particular, let x ∈ C^{N} be a vectorized multi-dimensional image with dimension N and let c ∈ C^{K} be a compressed representation of x with dimension K<N, where C denotes the complex numbers. Then c is a sliceable compressed representation (SCR) if any two-dimensional, potentially oblique, slice s ∈ C^{N} of the image x can be decompressed from a subset of c, denoted as cₛ ∈ C^{k}^{ₛ} with kₓ<K.

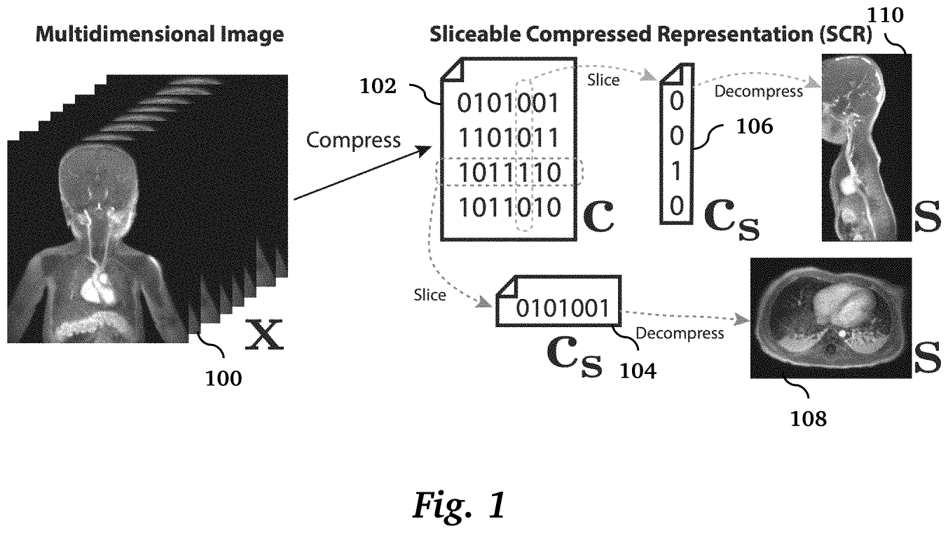

FIG. 1 illustrates an overview of processing pipeline of sliceable compressed representations used in embodiments of the invention. A multidimensional image 100 is acquired and compressed during reconstruction to produce a sliceable compressed representation 102, which is stored on a digital storage medium. During visualization, different slices may be selected, which are typically two-dimensional and may be oblique. The slices determine subsets 104, 106 of the stored sliceable compressed representation 102. The subsets 104, 106 are decompressed to produce image slices 108, 110, respectively, for display.

We consider situations where high-dimensional images are represented using SCR. High dimensional datasets can either be converted to SCR or reconstructed directly into SCR. We here consider datasets high-dimensional if they consist of more than two (spatial) dimensions. Examples of such additional dimension include slice, cardiac-phase and temporal dimensions.

Low Rank Representation

One example of SCR is the low rank representation for dynamic datasets. In particular, for a dynamic dataset with T frames, low rank representations produce spatial basis vectors (L ∈ C^{N'K}) and temporal basis vectors (R ∈ C^{T×K}), such that they are related to the underlying dynamic image series X=[x₁, . . . , x_{T}] by,

X=LR^{H}. (1)

By having a small rank K, low rank representations achieve a high compression ratio of NT/((N+T)K). We note that by defining the support of the basis vectors appropriately, the above relationship includes general structured low rank models, such as local low rank [8] and multi-scale low rank [7]. We consider a representation low rank if the matrix rank is less than N or T.

Low rank compressed representation allows access to a particular slice efficiently without ever operating on the full image. In particular, to obtain a slice indexed by a normal vector n of the plane and frame t, we can slice the spatial basis vector with respect to n (Lₙ) and the temporal basis vector with respect to t (Rₜ) independently and form the slice s as follows:

s=Lₙ Rₜ^{H}. (2)

The above operation is applied in general to low rank tensor representations, in which each factor matrices can be sliced independently. L and R are computed differently depending on the LR modeling methods mentioned above, but generally involve minimizing some objective function to approximate the original dataset.

Reconstruction Pipeline

According to embodiments of the present invention, SCR is enforced during reconstruction such that the full image is never created or stored. In particular, SCR is directly enforced in model-based reconstruction. Instead of storing the full images as DICOMs after reconstruction, we propose storing the SCR directly.

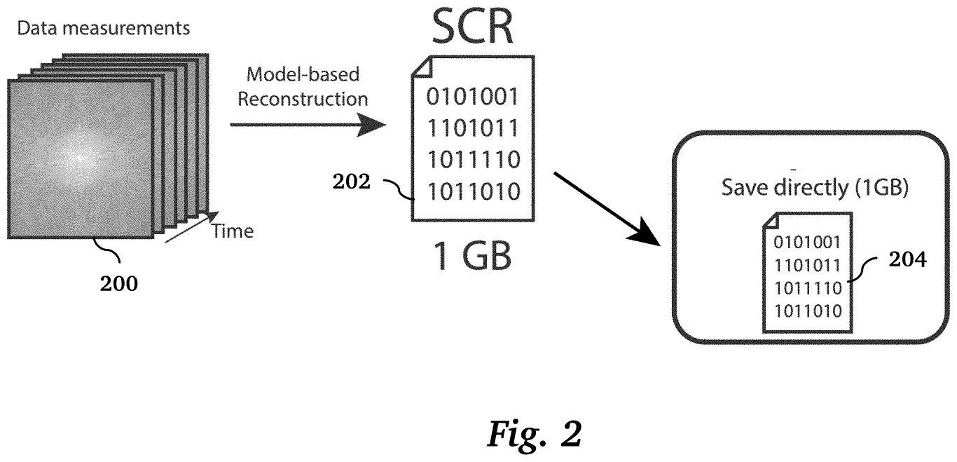

FIG. 2 is an illustration of a reconstruction pipeline according to an embodiment of the invention. Acquired data measurements 200 from a medical imaging scanner are input to a model-based reconstruction technique that produces a sliceable compressed representation 202.

For example, in low rank dynamic MRI reconstruction, we obtain data measurements y=A(X), where A represents the linear measurement system and X represents the underlying dynamic image series. Then explicit low rank representation can be enforced by solving

min_{L,R}∥y−A(LR^{H})∥₂²+g(L, R) (3)

where g is an optional regularization on the basis vectors L and R. The reconstruction then outputs the basis vectors L and R directly without ever expanding to the full-size image.

In another example, T2 shuffling uses a locally low rank regularization term for its reconstruction problem [6].

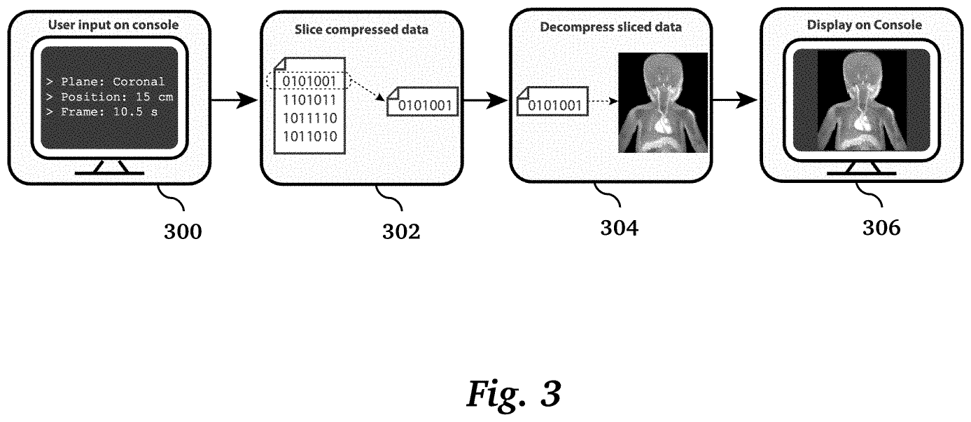

Visualization Pipeline Using properties of SCR, we can then efficiently reformat and visualize oblique slices without ever decompressing the image fully. As illustrated in FIG. 3, the visualization pipeline begins with a user (e.g., radiologist) specifying and inputting to the viewing console 300 which slice they want to visualize, e.g., by selecting a plane orientation, plane position, and plane time frame. Then the slice is recovered in step 302 by taking a subset cₛ of the compressed datasets. The sliced compressed representation cₛ is loaded to memory and decompressed in step 304 to form the slice s. Finally, in step 306, the slice is displayed on the viewer.

Experiment

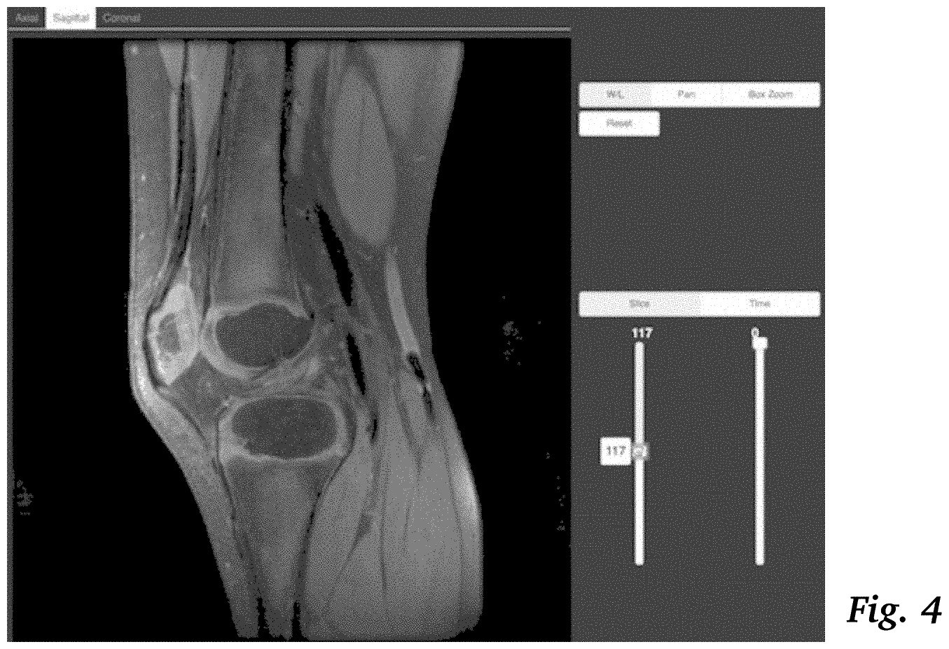

For purposes of illustration, we now describe an experiment. For visualization, we built a viewer capable of accessing and interacting with low rank reconstructed datasets according to the invention. The viewer uses a Python based interactive visualization library, Bokeh. The application code for the viewer is hosted on a server, which creates application instances upon request. This architecture allows for simple deployment for a network of users who can directly interact with the application. We demonstrated our visualization software on a knee MRI dataset reconstructed with T2 Shuffling. The dataset when uncompressed has a matrix size of 260×240×288 with 80 timestamps, and requires 10.8 GBs to store. In contrast, the compressed representation only used 660 MB. Loading the uncompressed data took 10 s, whereas our proposed method took 0.5 s on a laptop. The visualized image produced from the compressed representation is shown in FIG. 4.

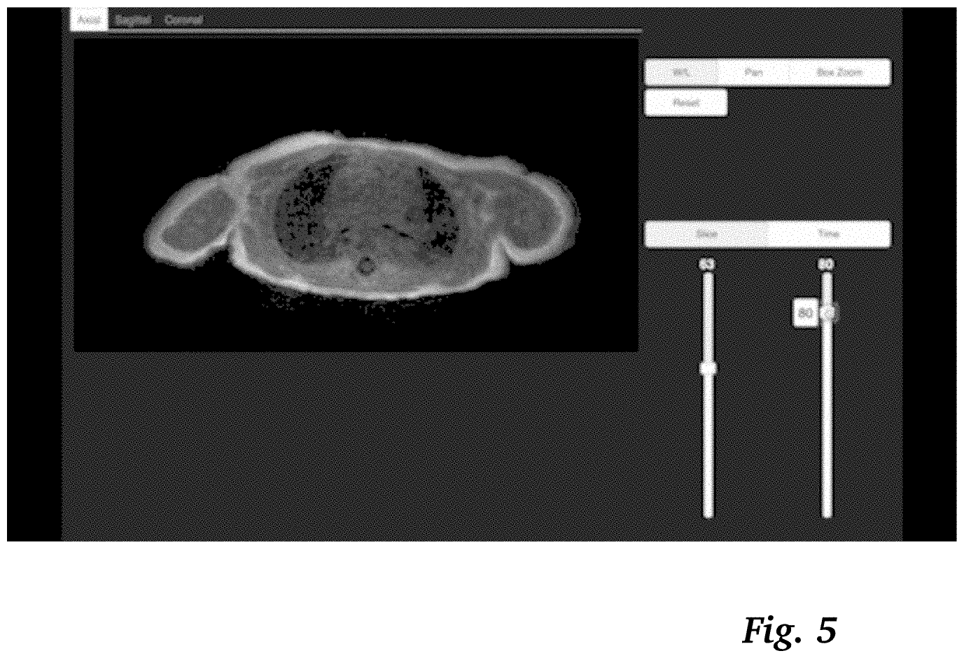

We also applied the proposed method on a chest DCE MRI dataset reconstructed with the multi-scale low rank reconstruction method (FIG. 5). The dataset if uncompressed has a matrix size of 323×186×332 with 500 timestamps and takes 74 GBs, which cannot be load in common viewer consoles. In contrast, the compressed representation only took 1.7 GBs and can be reformatted in real-time. The visualized image produced from the compressed representation is shown in FIG. 5.

Applications

This invention can be used for any multi-dimensional medical imaging applications with more than two dimensions. This invention would help reduce rate of memory storage and transfer and speed up visualization.

Embodiments of the invention may be implemented using a medical imaging device capable of acquiring high-dimensional datasets. In addition, it uses computers capable of handling the reconstruction and visualization workloads, as are used for other techniques.

### REFERENCES

[1] D. Donoho, “Compressed sensing,” IEEE Transactions on Information Theory, vol. 52, no. 4, pp. 1289-1306, April 2006.

[2] E. Candes, J. Romberg, and T. Tao, “Robust uncertainty principles: exact signal reconstruction from highly incomplete frequency information,” IEEE Transactions on Information Theory, vol. 52, no. 2, pp. 489-509, February 2006.

[3] R. L. Joshi, “System and method for rendering an oblique slice through volumetric data accessed via a client-server architecture,” U.S. Pat. No. 7,502,501, March 2009.

[4] S. G. Deshpande and W. Zeng, “Methods and systems for transmitting digital images,” U.S. Pat. No. 7,206,804, April 2007.

[5] K. Krishnan, M. W. Marcellin, M. S. Nadar, and A. Krishnan, “Prioritized image visualization from scalable compressed data,” U.S. Pat. No. 7,324,695, January 2008.

[6] J. I. Tamir, M. Uecker, W. Chen, P. Lai, M. T. Alley, S. S. Vasanawala, and M. Lustig, “T 2 shuffling: Sharp, multicontrast, volumetric fast spin-echo imaging: T 2 Shuffling,” Magnetic Resonance in Medicine, vol. 77, no. 1, pp. 180-195, January 2017.

[7] F. Ong and M. Lustig, “Beyond Low Rank+Sparse: Multiscale Low Rank Matrix Decomposition,” IEEE Journal of Selected Topics in Signal Processing, vol. 10, no. 4, pp. 672-687, June 2016.

[8] J. Trzasko and A. Manduca, “Local versus Global Low-Rank Promotion in Dynamic MRI Series Reconstruction,” in Proc. Intl. Soc. Mag. Reson. Med. 19, Montreal, Quebec, Canada, May 2011, p. 4371.

1. A method of medical imaging comprising:
performing a medical imaging scan to produce acquired imaging data;
reconstructing from the acquired imaging data a multi-dimensional medical imaging dataset in the form of a sliceable compressed representation, where the reconstruction does not at any stage create full decompressed images; and
producing from the sliceable compressed representation a selected image slice by decompressing only a subset of the sliceable compressed representation.

2. The method of claim 1 wherein producing from the sliceable compressed representation a selected image slice comprises selecting the subset of the sliceable compressed representation, and decompressing the subset to form the selected image slice.

3. The method of claim 1 further comprising storing the sliceable compressed representation in a lossless format.

4. The method of claim 1 further comprising displaying the selected image slice on a viewer for visualization.

5. The method of claim 1 wherein the sliceable compressed representation comprises a wavelet transform compressed representation or a low rank matrix and tensor based compressed representation.

6. The method of claim 5 wherein the low rank matrix and tensor based compressed representation is reconstructed using a T2 shuffling method or a multi-scale low rank method.
